(19) United States
(12) Patent Application Publication
(10) Pub. No.: US 2019/0125956 A1
(43) Pub. Date: May 2, 2019

(54) Treatment for Athersclerosis

(71) Applicant: Marv Enterprises, LLC, Hermitage, PA (US)

(72) Inventor: Mitchell S. Felder, Hermitage, PA (US)

(21) Appl. No.: 15/818,224

(22) Filed: Nov. 20, 2017

Related U.S. Application Data
(63) Continuation of application No. 14/376,549, filed on Aug. 4, 2014, now abandoned.
(60) Provisional application No. 61/616,200, filed on Mar. 27, 2012.

(51) Int. Cl. A61M 1/36; A61K 35/14; C07K 16/18

(52) U.S. Cl. CPC A61M 1/362; A61K 35/14; C07K 16/18

(57) ABSTRACT

A method is described to treat atherosclerosis by extracorporeally treating a patient's blood. A patient's blood is treated extracorporeally using a moiety that targets an antigen in the blood. The moiety facilitates removal of the blood antigen. The cleansed blood is then returned to the patient.

### CROSS-REFERENCE TO RELATED APPLICATIONS

This application claims benefit under 35 U.S.C. § 119(e) of U.S. Patent Application No. 61/616,200, filed Mar. 27, 2012, which is hereby incorporated herein by reference in its entirety.

### FIELD OF THE INVENTION

The present invention relates to an extracorporeal treatment for atherosclerosis.

### BACKGROUND OF THE INVENTION

In the United States, approximately 300,000 people a year die from heart disease or stroke. These two maladies produce the majority of hospital admissions and increase the cost of medical care. A substantial factor causing heart disease and strokes is the process of atherosclerosis. Atherosclerosis is a chronic disease that can remain asymptomatic for decades. Atherosclerosis can progress slowly, but is cumulative and often fatal. A common outcome resulting from widespread atherosclerosis is thrombosis of a coronary artery causing myocardial infarction. In a cerebral artery, atherosclerosis can cause strokes. Many other diseases share atherosclerosis as a major contributing factor. The alleviation of atherosclerosis would therefore be extremely useful in decreasing morbidity and mortality in patients.

### SUMMARY OF THE INVENTION

The present invention relates to a method of extracorporeal treating a patient's cerebrospinal fluid (blood). U.S. Ser. No. 13/128,870, U.S. Ser. No. 13/128,177, U.S. Ser. No. 13/254,855, and U.S. 61/612,474 are hereby incorporated by reference. The treatment includes a plurality of stages comprising removing blood from a patient, applying an extracorporeal treatment to the blood, and returning the blood to the patient.

In the first stage of the treatment, the blood is removed from the patient. A convenient method for removing blood includes standard venipuncture. In the second stage, a treatment is applied to the blood. The treatment can include an antibody directed against targeted antigens. The third stage comprises returning the blood to the patient and can also include removing the treatment from the blood.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

The method of the present invention comprises treating a patient's blood extracorporeally with an antibody designed to react with a particular targeted antigen (TA): adipocyte fatty acid-binding protein (A-FABP, FABP4), NF-κB (nuclear factor kappa-light-chain-enhancer of activated B cells), cyclooxygenase-2, interleukin-6 (IL-6), interleukin-1 (IL-1), resistin (adipose tissue-specific secretory factor (ADSF), C/EBP-epsilon-regulated myeloid-specific secreted cysteine-rich protein), tumor necrosis factor-alpha (TNF-α), and IKB kinase beta (IKKB, IKK2). The antibody can include a moiety, for example, an albumin moiety, that can complex with the target antigen/TA and thereby permit efficacious dialysis of the antibody-antigen complex. Dialysis methods are well known by one skilled in the art.

In an embodiment of the invention, the antibody comprises an albumin moiety and targets the removal of one or more TAs from the blood.

The target antigens can include adipocyte fatty acid-binding protein (A-FABP, FABP4), NF-κB (nuclear factor kappa-light-chain-enhancer of activated B cells), cyclooxygenase-2, interleukin-6 (IL-6), interleukin-1 (IL-1), resistin (adipose tissue-specific secretory factor (ADSF), C/EBP-epsilon-regulated myeloid-specific secreted cysteine-rich protein), tumor necrosis factor-alpha (TNF-α), and IKB kinase beta (IKKB, IKK2) can be differentiated using standard ELISA methodology. ELISA (enzyme-linked immunosorbant assay) is a biochemical technique that allows for the detection of an antigen in a sample. In ELISA, an antigen is affixed to a surface, and then an antibody is used for binding to the antigen. The antibody is linked to an enzyme which enables a color change in the substrate.

Other strategies may be employed to validate the level of target antigen in the body fluid: Western blotting technology, UV/vis spectroscopy, mass spectrometry, and surface plasmon resonance (SPR).

An alternative methodology of the present intervention would use a designer antibody with an attached macromolecular moiety instead of an albumin moiety. The macromolecular moiety, attached to the antibody, would be 1.000 mm to 0.005 mm in diameter. The antibody-macromolecular moiety-targeted antigen complex would then be blocked from reentering the patient's blood and/or body fluid circulation, by using a series of microscreens which contain openings with a diameter 50.0000% to 99.9999% less than the diameter of the designer antibody-macromolecular moiety. The microscreen opening(s) must have a diameter of at least 25 micrometers in order to allow for the passage and return to circulation of the non-pathological blood constituents.

In another alternative embodiment, the target antigens/TA, for example, TNF-α (tumor necrosis factor-alpha) and interleukin-6 (IL-6), may be captured by using antibody microarrays that contain antibodies to the targeted antigen(s). The antibody microarrays are composed of millions of identical monoclonal antibodies attached at high density on a surface, such as on a glass or plastic slide. After sufficient extracorporeal exposure of the TAs to the antibody microarrays, the antibody microarrays-TA may be disposed of using standard medical practice.

In still another alternative methodology, the intervention comprises removing the targeted antigen (s)/TAs: adipocyte fatty acid-binding protein (A-FABP, FABP4), NF-κB (nuclear factor kappa-light-chain-enhancer of activated B cells), cyclooxygenase-2, interleukin-6 (IL-6), interleukin-1 (IL-1), resistin (adipose tissue-specific secretory factor (ADSF), C/EBP-epsilon-regulated myeloid-specific secreted cysteine-rich protein), tumor necrosis factor-alpha (TNF-α), and IKB Kinase Beta (IKKB, IKK2) from the blood by using a designer antibody containing an iron (Fe) moiety. This will then create an Fe-antibody-antigen complex. This iron-containing complex may then be efficaciously removed using a strong, localized magnetic force field.

In another embodiment, immunoaffinity chromatography may be employed in which a heterogeneous group of molecules in the blood will undergo a purification process using entrapment on a solid or stationary phase or medium. Only the targeted antigens (TAs) will be trapped using immunoaffinity chromatography. A solid medium can be removed from the mixture, washed, and the TA(s) may then be released from the entrapment through elution.

Alternatively, gel filtration chromatography may be used in which the blood is used to transport the sample through a size exclusion column that will be used to separate the target antigen(s)/TA(s) by size and molecular weight.

In still another embodiment, the intervention filters using a molecular weight cut-off. Molecular weight cut-off filtration refers to the molecular weight at which at least 80% of the target antigen(s)/TA(s) is prohibited from membrane diffusion.

An article for performing the method can comprise at least three stages including a first stage, a second stage and a third stage. The first stage comprises removing blood from a patient. Removal can occur using any convenient method including, for example, a spinal tap. The second stage treats the blood. The third stage returns the treated blood to the patient after having achieved the physical removal of the targeted antigen(s)/TA(s): adipocyte fatty acid-binding protein (A-FABP, FABP4), NF-κB (nuclear factor kappa-light-chain-enhancer of activated B cells), cyclooxygenase-2, interleukin-6 (IL-6), interleukin-1 (IL-1), resistin (adipose tissue-specific secretory factor (ADSF), C/EBP-epsilon-regulated myeloid-specific secreted cysteine-rich protein), tumor necrosis factor-alpha (TNF-α), and IKB kinase beta (IKKB, IKK2).

After removal of the targeted antigen, the cleansed blood can then be returned to the patient, such as, for example by using the same catheter that was originally used in removing the blood. In one embodiment, the treatment of blood comprises removing about 25 mL to about 500 mL of blood from a patient, and then applying the treatment to the blood before returning it to the patient. The frequency of such treatments would depend upon an analysis of the underlying symptomatology and pathology of the patient.

An article for performing the method can contain one or more modules. A first module includes an inlet for blood and at least one exterior wall defining a treatment chamber. The treated blood can be returned to a patient, or the treatment chamber can be fluidly connected to a second module. The second module can comprise a removal module and an outlet for the blood for return to the patient. In embodiments, the removal module is selected from a group comprising a mechanical filter, a chemical filter, a dialysis machine, a molecular filter, molecular adsorbant recirculating system (MARS), a plasmapharesis unit, or combinations thereof.

The method includes removing blood from a patient in a first stage, treating the blood to obtain a reduction in the target antigen(s), and optionally removing the treatment from the blood in a second stage, and returning the blood to the patient in a third stage. The blood can be removed from the patient using any convenient method, including standard venipuncture procedure. The second stage can include sequentially passing the extracorporeal bodily fluid through a treatment chamber and a removal module.

The second stage applies a treatment to the blood, which can include introducing a designer antibody that joins with a targeted antigen (TA) in the blood to form an antibody-antigen complex. The antibody-antigen complex can be removed from the blood in the removal module. Optionally, the antibody-antigen complex can be conjugated with a second antibody comprising a moiety that increases the efficacy of removal to form an antibody-moiety-antigen complex.

In the third stage, the purified blood (body fluid with removed TA(s) : Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2), is returned to the patient.

An article for performing the method can comprise a first module including an inlet for blood and at least one exterior wall defining a treatment chamber that is fluidly connected to a second module comprising a removal module and an outlet for the body fluid to be treated. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal blood. The removal module can be any device capable of removing the antibody-antigen complex. In embodiments, the removal module is selected from a group comprising a mechanical filter, a chemical filter, a dialysis machine, a molecular filter, molecular adsorbant recirculating system (MARS), a plasmapharesis unit, or combinations thereof.

In an example, the first module of the article applies a treatment of an antibody with an attached albumin moiety that targets the antigen(s)/TA(s): Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1(IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2). The second module includes substantial removal of the treatment from the extracorporeal blood.

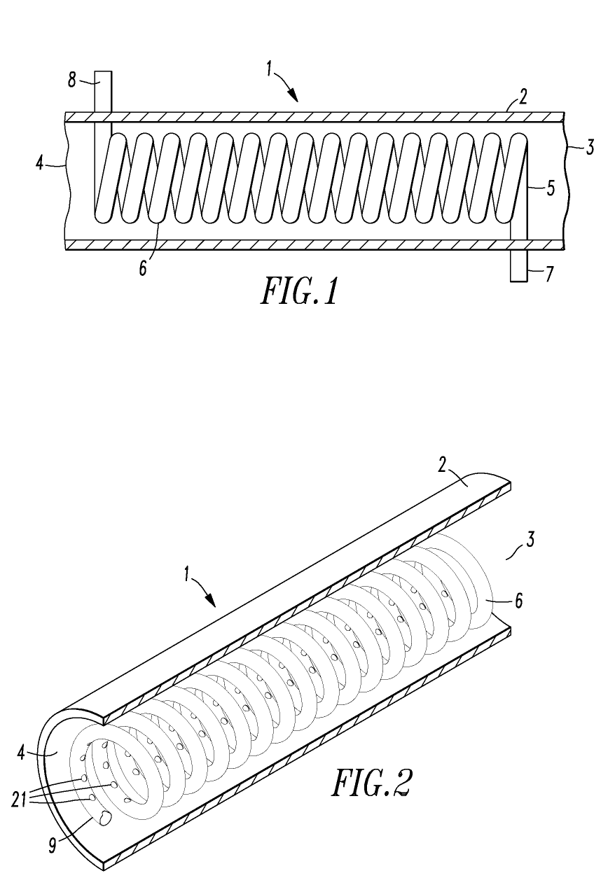

As shown in FIG. 1, the first module can include an exterior wall 2 defining a treatment chamber 5. The treatment can be applied in the treatment chamber 5. Residence times of the blood to be treated can be altered by changing the dimensions of the treatment chamber or the flow rate of the blood through the treatment chamber 5. Blood to be treated enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the blood. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer antibodies in the blood. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety, targeting the antigen/TA(s): Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2) can be delivered in a concurrent or counter-current mode with reference to the blood. In counter -current mode, the body fluid enters the treatment chamber 5 at the inlet 3. The designer antibody can enter through a first lead 8 near the outlet 4 of the treatment chamber 5. Blood then passes to the outlet 4 and the designer antibodies pass to the second lead 7 near the inlet 3. The removal module of the second stage substantially removes the designer antibodies-antigen molecular compound from the blood.

The second module can include a filter, such as a dialysis machine, which is known to one skilled in the art. The second module can include a molecular filter. For example, molecular adsorbents recirculating system (MARS), which may be compatible and/or synergistic with dialysis equipment. MARS technology can be used to remove small to average sized molecules from the blood. Artificial liver filtration presently uses this technique.

The method can include a plurality of steps for removing the targeted antigen(s)/TA(s): Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2). A first step can include directing a first antibody against the targeted antigen. A second step can include a second antibody. The second antibody can be conjugated with albumin, or alternatively a moiety which allows for efficacious dialysis. The second antibody or antibody-albumen complex combines with the first antibody forming an antibody-antibody-moiety complex. A third step is then used to remove the complex from the blood. This removal is enabled by using dialysis and/or MARS. The purified blood can then be returned to the patient.

In practice, a portion of the purified blood can be tested to ensure a sufficient portion of the targeted antigen(s)/TA (s) : Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2) has been successfully removed from blood. Testing can determine the length of treatment and evaluate the efficacy of the sequential dialysis methodology in removing the targeted antigens. Blood with an unacceptably large concentration of complex remaining can then be refiltered before returning the body fluid to the patient.

In embodiments, the second stage to remove the antibody-moiety-targeted antigen complex by various techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the antigen(s)/TA(s) : Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2).

Additional embodiments can include continuous renal replacement therapy (CRRT) which can remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming bodily fluid. A SC close to zero implies that the moiety antibody-targeted antigen complex will not pass through the filter. A filtration rate of 10 ml per minute is generally satisfactory. Other methods of increasing the removability of the moiety-antibody-targeted antigen include the use of temporary acidification of the bodily fluid using organic acids to compete with protein binding sites.

Embodiments of the present invention include a method for treating atherosclerosis comprising:
a. removing blood from a patient in a first stage;
b. applying a treatment to the blood in a second stage; and
c. returning the blood to the patient in a third stage.

Embodiments of the present invention also include such a method for treating atherosclerosis wherein the treatment includes
a. introducing an antibody that joins with an antigen in the blood to form an antibody-antigen complex; and
b. removing the complex from the blood.

Embodiments of the present invention also include such a method for treating atherosclerosis wherein the targeted antigen is selected from the group consisting of adipocyte fatty acid-binding protein (A-FABP, FABP4), NF-κB (nuclear factor kappa-light-chain-enhancer of activated B cells), cyclooxygenase-2, interleukin-6 (IL-6), interleukin-1 (IL-1), resistin (adipose tissue-specific secretory factor (ADSF), C/EBP-epsilon-regulated myeloid-specific secreted cysteine-rich protein), tumor necrosis factor-alpha (TNF-α), and IKB kinase beta (IKKB, IKK2), and combinations thereof.

Embodiments of the present invention also include a such method for treating atherosclerosis wherein the treatment includes
a. introducing a targeted antibody that joins with an antigen in the blood to form an antibody-antigen complex;
b. conjugating the antibody-antigen complex with a second antibody comprising a moiety that increases efficacy of removal to form an antibody-moiety-antigen complex; and
c. removing the complex from the blood.

Embodiments of the present invention also include such a method for treating atherosclerosis wherein the method includes testing the blood after the treatment and before returning the blood to the patient in order to determine the efficacy of treatment.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

All documents, books, manuals, papers, patents, published patent applications, guides, abstracts and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for treating atherosclerosis comprising:
a. removing blood from a patient in a first stage;
b. applying a treatment to the blood in a second stage; and
c. returning the blood to the patient in a third stage.

2. The method of claim 1, wherein the treatment includes
a. introducing an antibody that joins with an antigen in the blood to form an antibody-antigen complex; and
b. removing the complex form the blood.

3. The method of claim 2, wherein the targeted antigen is selected from a group consisting of Adipocyte fatty acid-binding protein (A-FABP, FABP4), NF-KB (Nuclear factor kappa-light-chain-enhancer of activated B cells), cyclooxygenase-2, Interleukin-6 (IL-6), Interleukin-1 (IL-1), Resistin (adipose tissue-specific secretory factor (ADSF), C/EBP-epsilon-regulated myeloid-specific secreted cysteine-rich protein), Tumor necrosis factor-alpha (TNF-alpha), and IKB Kinase Beta (IKKB, IKK2), and combinations thereof.

4. The method of claim 1, wherein the treatment includes
a. introducing a targeted antibody that joins with an antigen in the blood to form an antibody-antigen complex;
b. conjugating the antibody-antigen complex with a second antibody comprising a moiety that increases efficacy of removal to form an antibody-moiety-antigen complex; and
c. removing the complex from the blood.

5. The method of claim 4, wherein the method includes testing the blood after the treatment and before returning the blood to the patient in order to determine efficacy of treatment.
